(19) United States
(12) Patent Application Publication
(10) Pub. No.: US 2021/0236408 A1
(43) Pub. Date: Aug. 5, 2021

(54) METHOD FOR THE TREATMENT AND/OR PREVENTION OF DAMAGED KERATIN

(71) Applicants: Ranjithkumar RAVICHANDRAN, Linkoping (SE); Petter SIVLER, Linkoping (SE)

(72) Inventors: Ranjithkumar RAVICHANDRAN, Linkoping (SE); Petter SIVLER, Linkoping (SE)

(21) Appl. No.: 17/048,889

(22) PCT Filed: Apr. 19, 2019
(86) PCT No.: PCT/IB2019/053271

§ 371 Date: Oct. 19, 2020

(30) Foreign Application Priority Data
Apr. 26, 2018 (IT) 102018000004884

(51) Int. Cl. A61K 8/81; A61Q 5/00; A45D 19/00

(52) U.S. Cl. CPC A61K 8/8147; A61K 8/8117; A61Q 5/002; A45D 19/0066; A61K 2800/43; A61K 2800/438; A61K 2800/81; A61K 2800/95; A61K 2800/434

(57) ABSTRACT

A method is described for the treatment and/or prevention of damaged keratin in a keratin-containing tissue, comprising the steps of: applying on the keratin-containing tissue a cosmetic composition comprising molecules carrying at least one light reactive functional group, and at least one photoinitiator; and leaving to process with exposure of the keratin-containing tissue to light, the light being of a wavelength suitable for activating said at least one photoinitiator. A cosmetic composition is also described comprising molecules carrying at least one light reactive functional group and at least one photoinitiator; and its use.

### FIELD OF APPLICATION

The present invention pertains to the cosmetic sector and refers to a method for the treatment and/or prevention of damaged keratin, more particularly in hair, skin and/or nails.

### PRIOR ART

Keratin is a fibrous structural protein, making up hair, nails and the outer layer of human skin. Keratin monomers assemble to form intermediate filaments and have a structure exhibiting a central 310 circa residue domain with four segments in alpha-helical conformation that are separated by three short linker segments in beta-turn conformation. The keratin molecules supercoil to form a stable, left-handed superhelical motif to multimerise, forming filaments consisting of multiple copies of the keratin monomer.

Keratin contains many cysteine residues, which are rich in sulphur-containing functional groups, namely thiol groups (—SH) that form covalent bonds with other thiol groups in sulphur-containing functional groups on adjacent filaments. These strong crosslinks, which involve two sulphur atoms (S—S) are known as the disulphide bonds.

Human hair is 14% cysteine. Depending on the frequency of such disulphide bonds and their distribution on the hair, they contribute to the bending of hair, and hence to its shape, as well as to its texture. These covalent bonds resist in the presence of water and as a result wet hair resumes its original shape when dried.

Other weaker bonds also exist between the keratin filaments, such as Van der Waals bonds, hydrogen bonds, and Coulombic interactions. In contrast with the stronger disulphide bonds, these bonds are cleaved by water. New bonds are then formed upon evaporation of water. This is why, for example, wet hair is easier to style than dry hair.

Hair is a filamentous biomaterial, 20-180 μm in width, consisting mainly of keratin. The structure of a human hair shaft consists of the medulla at the centre of the hair, surrounded by the cortex, which contains keratins (40-60 kDa) and lipids. The cortex is surrounded by the cuticle, the outermost layer made of dead cells in a scale-like formation, that protects the hair. In healthy-looking hair, the cuticle has a smooth and glossy appearance. This is because the cuticle layer is tightly closed on the hair shaft (see FIG. 2b). The surface of the hair is covered in a covalently bound 18-methyl eicosanoic acid.

The keratin-containing cortex makes up most of the hair. It consist of loosely packed spindle-shaped cortical cells filled with keratin filaments orientated parallel to the length of the hair and an amorphous matrix of high sulphur proteins.

The cuticle may be damaged (or “weathered”) by mechanical forces, like the friction involved in hair brushing, or chemically, for example by oxidation, typical of bleaching procedures, or permanent styling. Excessive sunlight and heat also cause damage. Damaged hair usually exhibits an open cuticle, where the cuticle lifts from the hair shaft and can develop cracks and peel off (see FIG. 2a).

Damaged hair suffers from a decrease in lipid content, affecting its hydrophobicity, and is more porous than healthy looking hair. Damaged hair is also weak, and prone to premature fracturing, longitudinal splitting (i.e. split ends), separation of hair cortex, loss of strength and loss of gloss. This damage ultimately leads to cleaving of the cysteine di-sulphide bonds leading to the exposure of the free thiols.

As for skin and nails, damage to the disulphide bonds of the keratin causes the skin to look dry and unhealthy and nails to break easily.

Hair conditioners have been devised to counteract damage to hair and improve hair gloss. Their main action is in re-establishing the hydrophobicity of the hair shaft and eliminating negative charges on the hair fibre (responsible for static flyaway hair), by providing an oily coating on the cuticle. Conditioners normally contain polymers (typically poly-quaternium polymers), oils, waxes, hydrolysed aminoacids and/or cationic molecules. However, conditioners can also make the hair too greasy and prone to attract dirt. Moreover, any beneficial effect of the conditioner is only temporary, as the oily coating is washed off by surfactants with each shampoo.

There is therefore a need in the field for a method and a cosmetic composition that enable to treat and/or prevent damaged keratin, in particular in hair, skin and/or nails, in an effective and long lasting way.

The technical problem underlying the present invention is thus that of making available a method, as well as a composition, for the treatment and/or prevention of damaged keratin, in particular in hair, skin and/or nails, more particularly in hair, even more particularly in human scalp hair, that has a long lasting effect, for example an effect on hair lasting at least six months.

Another technical problem underlying the present invention is that of making available such a method and composition that do not leave undesired residues on the hair shaft, that can negatively affect its gloss and its silky appearance.

Another technical problem underlying the present invention is that of making available such a method and composition that also further improve the overall appearance of the hair, for example by improving moisture, thus preventing hair dryness, and by increasing hair volume.

Another technical problem underlying the present invention is that of making available such a method and composition that also help to protect and repair the hair from damage of a chemical or physical origin, such as for example from coloring, bleaching, straightening or styling techniques, thereby also facilitating the use of such techniques.

Another technical problem underlying the present invention is that of making available such a method and composition that can be easily incorporated into existing hair-colouring/dyeing and hairstyling techniques, both professionally (i.e. in hair saloons) and at home, as well as into hair care products, such as for example conditioners, hair dyeing/colouring products, and hair sprays).

Another technical problem underlying the present invention is that of making available such a method and a composition that are also environmentally friendly.

### SUMMARY OF THE INVENTION

Such a problem has been solved, according to the present invention, by a method for the treatment and/or prevention of damaged keratin in a keratin-containing tissue, comprising the steps of:
a) applying on the keratin-containing tissue a cosmetic composition comprising molecules carrying at least one light reactive functional group, and at least one photoinitiator; and
b) leaving to process with exposure of the keratin-containing tissue to light, the light being of a wavelength suitable for activating the at least one photoinitiator.

The above method of the present invention will also be referred to as the “first method”.

By “keratin-containing tissue” what is meant here is a body tissue that typically features the presence of keratin filaments with disulphide bonds, such as for example human or animal hair, human or animal skin and nails.

Preferably, the keratin-containing tissue is chosen from human or animal hair, human or animal skin and/or (human) nails, more preferably human or animal hair, even more preferably human scalp hair.

The term “hair” refers to one or more than one strand of hair.

Preferably, the cosmetic composition is a fluid composition, more preferably liquid.

In a preferred embodiment, the cosmetic composition is in the form of a suspension or an emulsion, more preferably a suspension.

The cosmetic composition can, for example, be in the form of a liquid, a lotion, a milk, a mousse, a spray, a gel, a cream, a shampoo or a conditioner.

Preferably, the cosmetic composition is applied through a squeeze bottle nozzle or by spraying, or by any other method known in the field, according to convenience.

By “light reactive functional group” what is meant here is a functional group that can be activated by light having a wavelength comprised between 10 nm and 100 μm, more preferably comprised between 300 nm and 1400 nm, even more preferably comprised between 360 nm and 800 nm, in the presence of a suitable photoinitiator, to form reactive intermediates capable of forming covalent bonds with the target sites, namely free thiols (also referred to as thiol groups).

In the present specification, the word “light” is used to mean “electromagnetic radiation”. Throughout the specification, the expression “electromagnetic radiation” and the word “light” are also referred to as “radiation”.

In the present invention, by ultraviolet (UV) radiation (or light) is meant radiation (or light) having a wavelength of 300 nm-400 nm; by visible radiation (or light) is meant radiation (or light) having a wavelength of >400 nm-700 nm, by near infrared (NIR) radiation (or light) is meant radiation (or light) having a wavelength of >700 nm-1400 nm.

Preferably, the light reactive functional groups are chosen from acrylates, methacrylates, vinyl benzene, alkynes, and combinations thereof, more preferably alkynes.

Preferably, the molecules carrying at least one light reactive functional group, wherein the functional group is acrylate, methacrylate or vinyl benzene, carry at least two said light reactive functional groups. Hereinafter, the expression “reactive molecules” will also be used to refer to “molecules carrying at least one light reactive functional group”. Similarly, “reactive molecule” will also be used to refer to a “molecule carrying at least one light reactive functional group”.

Preferably, the reactive molecules have a degree of substitution greater than 5%, more preferably greater than 20%, even more preferably greater than 30%, even more preferably greater than 40%, even more preferably greater than 50%, most preferably greater than 55%.

The degree of substitution can be modulated based on the molar ratio between the starting molecule and the substituting groups. By way of example, the starting molecule and the substituting groups are respectively gelatin and methacrylic anyhride (MAA), in the preparation of methacrylate-functionalised gelatin (see Example 1).

By the expression “degree of substitution” of a reactive molecule of the present invention what is meant here is the percentage number of functional groups (for example amines, carboxylic acids or hydroxyl groups) of a starting molecule that have been functionalised with said light reactive functional groups, based on the total number of functional groups (for example amines, carboxylic acids or hydroxyl groups) involved in the functionalisation reaction of said starting molecule. The degree of substitution can be determined by any method known in the art, such as for example NMR (Nichol., J. W. et al. (2010) Biomaterials, 31 (21) 5536-5544); the Trinitrobenzoic acid assay (Yoon, H. J. et al. (2016) PLoS ONE 11(10) 1-18); or the Habeeb method (Habeeb, A F. (1966) Anal. Biochem, 14, 328-336); preferably NMR.

In the NMR method, for example, a sample containing functionalised molecules is dissolved in deuterated solvents, for example D20 (Deuterated) or dDMSO (Deuterated Dimethyl Sulfoxide), and analysed by ¹H NMR spectroscopy using, for example, a 300 MHz Varian Spectrometer (Varian Inc, Palo Alto, Calif., US). Comparison of the results with a non-functionalised sample allows to determine the percentage number of the total potential functional groups that have been functionalised.

Preferably, the reactive molecules are obtained from the functionalisation of the backbone, side chain(s) and/or terminus/(i) of the starting molecules.

Preferably, the reactive molecules wherein the light reactive functional group is alkyne are obtained from the functionalisation of the backbone, side chain(s) and/or terminus/-i of the starting molecules, more preferably the side chain(s).

Preferably, the reactive molecules wherein the light reactive functional group is acrylate, methacrylate or vinyl benzene are obtained from the functionalisation of the side chain(s) of the starting molecules.

Preferably, in the reactive molecules, the light reactive functional group(s) is/are covalently bound.

Preferably, the reactive molecules are compatible with aqueous or solvent cosmetic delivery systems, more preferably aqueous cosmetic delivery systems.

Preferably, the reactive molecules of the present invention are chosen based on their chemical compatibility with the keratin-containing tissue, more preferably human or animal hair, even more preferably human scalp hair, most preferably also on their suitability for being rinsed off the keratin-containing tissue, typically with water, when in excess.

In a preferred embodiment of the present invention, the reactive molecules are obtained from the functionalisation of aminoacids, saccharides and/or fatty acids, and/or residues thereof. In other words, the starting molecules are preferably aminoacids, saccharides and/or fatty acids, and/or residues thereof.

Preferably, the aminoacids, saccharides and/or fatty acids, and/or residues thereof, are natural aminoacids, saccharides and/or fatty acids, and/or residues thereof. The expression “natural aminoacids, saccharides and/or fatty acids, and/or residues thereof” is used herein to refer to aminoacids, saccharides and/or fatty acids, and/or residues thereof, that are found in nature.

The aminoacid can be a free aminoacid or can be bound via a peptide bond to other aminoacids in the form of branched or linear polypeptide chains, or proteins.

Preferably, the branched or linear polypeptide chain has a chain length comprised between 2 and 3000 aminoacid residues.

Preferably, the proteins have a molecular weight comprised between 0.1 kDa and 800000 kDa, more preferably comprised between 1 kDa and 100000 kDa, even more preferably comprised between 2 kDa and 300 kDa.

Preferably, proteins are chosen from collagen, gelatin, keratin, and albumin.

In a preferred embodiment, proteins and aminoacids are chosen from plant proteins and aminoacids, that is, proteins and aminoacids from a plant source.

Preferably, the plant proteins are chosen from hydrolysed soy protein, hydrolysed wheat protein, hydrolysed rice protein, hydrolysed quinoa protein, hydrolysed barley protein, hydrolysed vegetable protein and mixtures thereof.

Preferably, the plant aminoacids are a mixture of aminoacids, the mixture mimicking the characteristics of keratin, such as for example solutions containing wheat and soy aminoacids (for example that under the trade name of Fision KeraVeg18 by TRI-K Industries Inc, India).

Examples of reactive molecules obtained from the functionalisation of proteins are gelatin methacryloyl, gelatin acrylate, gelatin methacrylamide/methacrylate, gelatin vinyl benzene, and glycidyl methacrylate-modified gelatin.

Functionalisation of proteins and/or residues therof can be carried out according to known methods in the field. In particular, functionalisation of proteins in their side chains with acrylates or methacrylates can be carried out according to known methods in the field, such as for example Nichol, J W. et al. (2010, Nichol, J W. et al. (2010) Biomaterials, 31(21):5536-5544); Gaudet I D. and Shreiber, D I. (2012, Gaudet I D. and Shreiber, D I. (2012) Biointerphases, 2012, 7:25); Brinkman, W T. et al. (2003, Brinkman, W T. et al. (2003) Biomacromolecules, 4, 890-895); Teramoto et al. (2012, Teramoto et al. (2012), Materials, 5, 2573-2585); Van Den Bulcke et al. (2000, Van Den Bulcke et al. (2000) Biomacromolecules, 1, 31-38).

Preferably, the saccharides are in a form chosen from monosaccharides, disaccharides, branched or linear oligosaccharides, and branched or linear polysaccharides.

Preferably, the polysaccharides have a molecular weight comprised between 0.1 kDa and 800000 kDa, more preferably comprised between 1 kDa and 300000 kDa, even more preferably comprised between 2 kDa and 300 kDa.

Preferably, the polysaccharides are chosen from, hyaluronic acid, alginates, dextrose, cellulose, and derivatives thereof. For example, the polysaccharide can be a salt, preferably a hyaluronic acid salt.

Preferably the monosaccharides are chosen from dextrose, glucose and fructose. Functionalisation of saccharides and/ore residues thereof can be carried out according to known methods in the field. In particular, functionalisation of saccharides with acrylates or methacrylates, or alkynes can be carried out according to known methods in the field, such as Smeds K A. et al. (2001, Smeds K A. et al. (2001) Journal of Biomedical Material Research, 54, 115-121); Fenn D. et al. (2009, Fenn D. et al. (2009) Reactive & Functional polymers 69, 347-352); Yin, R. et al. (2010, Yin, R. et al. (2010) Carbohydrate polymers, 82, 412-418); Roy, B. and Mukhopadhyay, B. (2007, Roy, B. and Mukhopadhyay, B. (2007) Tetrahedron Letters, 48, 3783-3787).

Preferably, the fatty acids are free fatty acids or bound to form a lipid, for example esterified with glycerol to form glycerides or phospholipids, or bound to form other lipids. The fatty acids can be in the form of saturated or unsaturated fatty acids.

Preferably, fatty acids have a number of carbon atoms comprised between 2 and 100 more preferably comprised between 5 and 70, even more preferably comprised between 7 and 25.

Preferably, the fatty acids are chosen from stearic acid, oleic acid, palmitic acid, lauric acid, linoleic acid.

Functionalisation of fatty acids and/or residues thereof can be carried out according to known methods in the field. In particular, functionalisation of fatty acids with acrylates, methacrylates or alkynes can be carried out according to known methods in the field, such as for example Adekunle, K. et al. (2009, Adekunle, K. et al. (2009) Journal of Applied Polymer Science, 115, 6, 3137-3145); Thiele, C. et al.(2012, Thiele, C. et al. (2012) ACS Chemical Biology, 7, 2004-2011); Tronci et al. (2013, Tronci et al. (2013) J. Mater. Chem. B. Mater. Biolo. Med., 1(30), 2705-3715), La Scala, J. J. et al. (2004, La Scala, J. J. et al. (2004) Polymer 45, 7729-7737).

Alternatively, or in addition, the reactive molecules are obtained from the functionalisation of synthetic polymers, more preferably synthetic polymers carrying at least one alkyne functional group. In other words, the starting molecules are preferably synthetic polymers, more preferably synthetic polymers carrying at least one alkyne functional group.

The synthetic polymers are chosen among those suitable for cosmetic uses, and comprise polyvinyl pyrrolidone homo and copolymers, polyvinyl acetate, polyacrylates, polymethacrylates, polyamides, polyvinylamides, polyurethanes, and silicones.

Preferably, the synthetic polymers are functionalised with alkyne functional groups. Examples of reactive molecules obtained from the functionalisation of synthetic polymers with alkynes are functionalised aminoacids or peptides, such as for example poly-L-lysine hydrobromide graft alkyne (with functionalisation either at the side chain or at the terminus) and L-homopropargylglycine.

Examples of reactive molecules obtained from the functionalisation of synthetic polymers with alkynes at the backbone, also referred to as ynamides, are benzenesulfonamides, oxazolidinone and pyrrolidinones, such as 4-methyl-N-(2-phenylethynyl)-N-(phenylmethyl) benzenesulfonamide, 3-(1-octyn-1-yl)-2-oxazolidinone, 3-(2-phenylethynyl)-2-oxazolidinone, 1-(2-phenylethynyl)-2-pyrrolidinone, (R)-4-Phenyl-3-(2-phenylethynyl)oxazolidin-2-one, and TMS-N-ethynyl-N,4-dimethylbenzenesulfonamide 95%.

Other examples of alkyne-functionalised synthetic polymers are alpha-methyl-poly(2-ethyl-2-oxazoline)-omega-alkyne (Me-PEtOx-alkyne), (S)-(−)-2-Azido-6-(Boc-amino)hexanoic acid (dicyclohexylammonium) salt, (S)-2-Azido-3-(4-tert-butoxyphenyl)propionic acid cyclohexylammonium salt, (S)-5-Azido-2-(Fmoc-amino)pentanoic acid, L-Azidohomoalanine hydrochloride, (S)-2 Azido-3-(3-indolyl)propionic acid cyclohexylammonium salt, (S)-2-Azido-3-methylbutyric acid cyclohexylammonium salt, (S)-2-Azido-3-phenylpropionic acid (dicyclohexylammonium) salt, (R)-2-(Boc-amino)-5-hexynoic acid, Boc-3-azido-Ala-OH (dicyclohexylammonium) salt, N-Boc-4-azido-L-homoalanine (dicyclohexylammonium) salt, N-Boc-6-azido-L-norleucine (dicyclohexylammonium) salt, Boc-4-azido-Phe-OH, Boc-propargyl-Gly-OH, Boc-(R)-4-(2-propynyl)-L-proline, (S)-(−)-4-tert-Butyl hydrogen 2-azidosuccinate (dicyclohexylammonium) salt, N2-[(1,1-Dimethylethoxy)carbonyl]-N6-[(2-propynyloxy)carbonyl]-L-lysine 90%, Fmoc-β-azido-Ala-OH, Fmoc-(R)-propargyl-Ala-OH, Fmoc-(S)-propargyl-Ala-OH, L-Homopropargylglycine hydrochloride.

The cosmetic composition of the present invention can comprise a mixture of any of the reactive molecules described above, namely reactive molecules obtained from the functionalisation of: aminoacids and/or residues thereof; saccharides and/or residues thereof; fatty acids and/or residues thereof; and synthetic polymers, in any combination thereof.

For example, the cosmetic composition may comprise reactive molecules obtained from the functionalisation of aminoacids and/or residues thereof and reactive molecules obtained from the functionalisation of synthetic polymers; or it may comprise reactive molecules obtained from the functionalisation of aminoacids and/or residues thereof and reactive molecules obtained from the functionalisation of saccharides and/or residues thereof; or it may comprise reactive molecules obtained from the functionalisation of aminoacids and/or residues thereof, reactive molecules obtained from the functionalisation of saccharides and/or residues thereof, and reactive molecules obtained from the functionalisation of synthetic polymers; or any other combination of the above mentioned reactive molecules.

The reactive molecules of the present invention can be synthesized according to methods known in the field. Also, the reactive molecules of the present invention may be commercially available.

Preferably, in the method of the present invention, the damaged keratin in a keratin-containing tissue comprises at least two free thiol groups, more preferably at least 2 μmoles of free thiol groups per gram of keratin-containing tissue, most preferably from to 1000 μmoles of free thiol groups per gram of keratin-containing tissue.

Preferably, in the method of the present invention, the keratin-containing tissue is human or animal hair, even more preferably human scalp hair, and it comprises at least 2 μmoles, more preferably at least 20 μmoles, even more preferably at least 200 μmoles, most preferably from 200 μmoles to 1000 μmoles, of free thiol groups per gram of keratin-containing tissue.

The determination of the concentration of the number of thiol groups per gram of keratin-containing tissue can be carried our according to any method known in the art, for example according to the method described in Xu et al. (2017), Green and sustainable technology for high-efficiency and low-damage manipulation of densely crosslinked proteins, ACS Omega, 2, 1760-1768, under the paragraph “Titration of Sulfhydryl Groups, Carboxyl Groups, and Amine Groups” under the Materials and Methods section.

Preferably, the damaged keratin-containing tissue is human or animal hair, more preferably human scalp hair, and the hair exhibits one or more of the following features: a dry texture, a dull appearance, poor gloss, weakness, fracturing, split ends, and reduced surface lipids.

Preferably, the damaged keratin-containing tissue is human or animal skin, and the skin exhibits one or more of the following features: dryness, itchiness, wrinkles, and reduced surface lipids.

Preferably, the damaged keratin-containing tissue is (human) nails, and the nails exhibit one or more of the following features: brittleness, porosity, softness, presence of white lines, and reduced surface lipids.

Preferably, in the cosmetic composition of step a) the reactive molecules are present in a concentration, in weight percent based on the volume of the composition, of at least 0.01%, more preferably comprised between 0.05% and 20%, even more preferably comprised between 0.1 and 15%, most preferably between 0.5 and 10%.

Preferably, in step a) the keratin-containing tissue is chosen from human or animal hair; human or animal skin; and/or (human) nails, more preferably human or animal hair, even more preferably human scalp hair, and the keratin comprises at least two free thiol groups. In other words, the keratin is damaged keratin.

Preferably, in step a) of the first method the keratin is damaged keratin.

Preferably, the animal hair and/or skin is mammal hair and/or skin, more preferably the hair and/or skin of pets, such as for example cats, dogs, rabbits and horses.

It is within the ability of the skilled person to adjust the amount of composition to be applied, and the concentration thereof, based for example on the extent of damage to the keratin and the desired result to be achieved.

Preferably, in step a) the keratin-containing tissue is human or animal hair, more preferably human scalp hair, and it is damp. Damp hair, in fact, allows better penetration of the reactive molecules, compared to dry hair.

A photoinitiator is a molecule that converts absorbed ultraviolet, visible or near infrared light into chemical energy in the form of initiating species (i.e. the photoinitiator is activated), for example free radicals or cations.

Preferably, the photoinitiator is activated (or excited) at a wavelength comprised between 10 nm and 100 μm, more preferably between 300 and 1400 nm, even more preferably between 360 nm and 800 nm, most preferably between 360 nm and 400 nm.

Preferably, the photoinitiator is a radical photoinitiator.

The at least one photoinitiator can be a one-component, two-component or multi-component system, preferably a one-component system.

Preferably, the photoinitiator is water-soluble. According to one embodiment of the invention, the photoinitiator is activated (or excited) upon UV radiation and is preferably chosen from alpha hydroxyketones or derivatives thereof, such as for example benzoin ethers, benzyl ketals, alpha-dialkoxy-alkyl-phenones, alpha-hydroxy-alkyl-phenones, alpha-amino-alkyl-phenones, acyl-phosphine oxides, benzo-phenones and thio-xanthones.

Examples of suitable photoinitiators in the UV light spectrum are bis(2,4,6-trimethylbenzoyl)-phenylphosphineoxide (Irgacure® 819), bis(.eta.5-2,4-cyclopentadien-1-yl)-bis(2,6-difluoro-3-(1H-pyrrol-1-yl)-phenyl)titanium (Irgacure® 784), 1-[4-(2-Hydroxyethoxy)-phenyl]-2-hydroxy-2-methyl-1-propane-1-one (Irgacure® 2959).

According to another preferred embodiment of the invention, the photoinitiator is activated (or excited) upon visible light. Examples of suitable photoinitiators in the visible light spectrum are 2′,4′,5′,7′ -Tetrabromofluorescein disodium salt (EosinY), 2,2′-Azobis[2-methyl-N-(2hydroxyethyl)propionamide(VA-086), monoacylphosphienoxide salt and bisacylphosphineoxide salt, titanocenes.

According to yet another preferred embodiment of the invention, the photoinitiator is activated (or excited) upon near IR radiation and is preferably chosen from cyclic benzylidene ketone-based initiators.

Preferably, the photoinitiator is present in the cosmetic composition in a concentration, in weight percent based on the total volume of the cosmetic composition, comprised between 0.01% and 10% more preferably comprised between 0.1% and 5%, even more preferably comprised between 0.5% and 3%.

Preferably, the cosmetic composition has a pH comprised between 4 and 9 more preferably comprised between 6 and 7.4.

Preferably, the cosmetic composition further comprises one or more eccipients chosen from water, phosphate buffer saline (PBS) and oil, preferably water.

Preferably, the water is ultrapure water, for example Milli Q® water (Millipore Corporation, Darmstadt, Germany).

Preferably, the cosmetic composition further comprises one or more ingredients, more preferably chosen from moisturisers, acidity regulators, oils, surfactants, lubricants, sequestrants, antistatic agents, antimicrobial agents, preservatives, emulsifiers, thickeners, emolients and sunscreen and/or fragrances.

Preferably, the cosmetic composition further comprises at least one colouring agent, preferably chosen from a pigment, a dye and a combination thereof. Preferably the colouring agent is present in the cosmetic composition in a concentration, in weight percent based on the volume of the cosmetic composition, comprised between 0.0001 and 1%, more preferably between 0.001 and 0.1%.

In a preferred embodiment, the colouring agent is capable of emitting light when exposed to radiation having a wavelength comprised between 10 nm and 100 μm, more preferably between 300 nm and 1400 nm, even more preferably between 360 nm and 800 nm, most preferably between 360 and 400 nm.

Preferably, the colouring agent is a fluorescent dye or a non-fluorescent dye, more preferably a fluorescent dye.

Fluorescein is an example of a fluorescent dye that can be used in the present invention.

Preferably, the colouring agent is physically or covalently bound to the molecules, more preferably physically bound, for example through Van der Waal's forces or hydrogen bonds.

Preferably, in step a) the cosmetic composition is applied evenly on the keratin-containing tissue.

Preferably, the keratin-containing tissue is human or animal hair, more preferably human scalp hair, and the cosmetic composition is applied with the aid of a brush or comb.

The presence of a colouring agent, in particular a fluorescent one, enables the user to determine, for example by exposing the hair to UV radiation, whether the cosmetic composition is evenly applied on the hair. It is known that mechanical stresses, such as excessive brushing of hair, especially when wet, leads to hair damage. Being able to determine when the composition is evenly applied thus has the dual advantage of optimising the uniformity of the result while at the same time avoiding any unnecessary handling of the hair once the composition is evenly spread.

In the present invention, the expression “leaving to process” of step b) means that the cosmetic composition is left in contact with the keratin-containing tissue.

Preferably, in step b) the composition is left to process for a processing time of at least 5 minutes, more preferably for a time comprised between 7 and 40 minutes, even more preferably for a time comprised between 10 and 30 minutes, most preferably for a time comprised between 15 and 20 minutes.

Preferably, in step b), the temperature of the environment surrounding the keratin-containing tissue is between 4 and 35° C., more preferably between 10 and 30° C., most preferably between 20 and 25° C.

Preferably, in step b) the keratin-containing tissue is exposed to a light having a wavelength comprised between 360 and 800 nm, more preferably comprised between 360 and 400 nm (UV light).

Preferably, in step b) the keratin-containing tissue is exposed to the light for at least 5 minutes, more preferably a time comprised between 7 and 40 minutes, even more preferably for a time comprised between 10 and 30 minutes, most preferably for a time comprised between 15 and 20 minutes.

Preferably, in step b) the keratin-containing tissue is exposed to the light for the duration of the processing time.

The keratin-containing tissue can be exposed to light using any known suitable light-emitting device known in the field, such as for example radiation lamps or radiation-emitting combs.

Preferably, in step b), the keratin-containing tissue is human or animal hair, more preferably human scalp hair, and the hair is combed or brushed, more preferably using a light-emitting comb or brush, even more preferably a UV-A comb or a laser comb.

Preferably, the light-emitting comb or brush emits light having a wavelength comprised between 360 nm and 400 nm, more preferably comprised between 360 and 380, even more preferably 370 nm.

Preferably, the light-emitting comb or brush emits both ultraviolet light and NIR light, preferably by switching between the ultraviolet light and NIR light during use.

Preferably, in step b) the hair is combed or brushed for the duration of the processing time.

Preferably, step b) is followed by step c) of rinsing the keratin-containing tissue, preferably the human or animal hair, more preferably human scalp hair.

Preferably, step c) of rinsing is carried out, with water, until complete removal of excess residues from the keratin-containing tissue, preferably unreacted reactive molecules.

As mentioned above, damaged keratin is characterised by the fact that disulphur (or rather, disulphide) bridges on and between keratin filaments are broken, thus exposing free thiols.

The present invention solves the technical problem by providing functional groups that covalently link to the free thiols to establish crosslinks between them.

This, in turn, re-establishes the smooth and healthy appearance typical of healthy-looking keratin-containing tissue, such as human or animal hair, nails and human or animal skin. Provided that the overall number of reactive molecules applied are in sufficient amount, all free thiols can be crosslinked in one application of the composition.

The reactive molecules of the present invention, in fact, when activated by the photoinitiator, enable the functional groups, namely acrylates, methacrylates, vinyl benzene, and/or alkynes, to covalently link to the free thiols on the damaged hair, thus forming crosslinks. This reaction is a radical-mediated thiol-ene (in the case of acrylates, methacrylates and vinyl benzene) or thiol-yne (in the case of alkyne) coupling.

An overview of the reactions involved in the crosslinking of alkenes and alkynes is provided in Konuray et al. (2018, Konuray et al. (2018) Polymers, 10, 178).

In the case of acrylate, methacrylate, or vinyl benzene functional groups, one alkene can crosslink with one free thiol. To establish a crosslink (i.e. bridge the broken disulphide) between two free thiol groups, at least two such functional groups are required on the same reactive molecule. On the other hand, in the case of alkynes, one such functional group on the reactive molecule suffices to bind to two thiols and bridge them.

FIG. 1 shows schematically the mechanism by which the method of the invention repairs damaged keratin, for example on hair.

In FIG. 1a, the schematic drawing on the left hand side of the arrow represents the keratin filaments of healthy hair, which exhibits naturally occurring disulphide bonds (or bridges). Following environmental or chemical stresses (represented by the arrow), the disulphide bonds are cleaved, giving rise to free thiols, as schematically represented on the right hand side of the arrow of FIG. 1a.

The method of the present invention provides reactive molecules that, in the presence of light, bind to the free thiols, thus establishing crosslinks between adjacent free thiols. This is shown in FIG. 1b, with particular reference to a reactive molecule carrying at least two acrylate or methacrylate functional groups. FIG. 1c shows the crosslinking between adjacent free thiols according to the method of the invention in the case of a reactive molecule carrying at least one alkyne functional group.

As the crosslinking is based on a strong covalent bond, the effect of the method is permanent, having a duration of at least 30 days, more preferably at least 6 months, even more preferably at least one year, most preferably at least 18 months.

In order to prevent further damage to the keratin, to treat any new keratin damage, and/or to maintain the result longer in time, in particular in human or animal hair, more preferably human scalp hair, the above-described method of the present invention can be repeated once or more on the previously treated keratin-containing tissue.

Accordingly, in the preferred embodiment of the present invention, steps a) to c) of the method of the present invention are repeated.

In the following, the repetition of the method of the present invention according to this preferred embodiment will also be referred to as the “second method”.

Preferably, in step a) of the second method, the cosmetic composition is applied on a keratin-containing tissue, more preferably human or animal hair, even more preferably human scalp hair, comprising keratin filaments covalently bound to said reactive molecules.

Preferably, the keratin-containing tissue, more preferably human or animal hair, even more preferably human scalp hair, comprising keratin filaments covalently bound to the reactive molecules has previously undergone the method for the treatment and/or prevention of damaged hair of the present invention (i.e. first method).

According to this embodiment of the invention, the cosmetic composition can be formulated to be a hair conditioning composition. This can be achieved for example with the addition of hair conditioning agents to the cosmetic composition of the invention, or by adding the cosmetic composition of the invention to an existing hair conditioning composition.

In the present invention, by “conditioning agent” is meant a cosmetic ingredient which is used in conditioning compositions to soften, smooth or change the sheen of the hair. Suitable conditioning agents include but are not limited to polycationic polymers, for example polyquaternium polymers or silicone quaternium-8; alcohols, for example oleyl alcohol, stearyl alcohol, cetyl alcohol, panthenol; oils, such as meadowfoam seed oil, mango seed oil, grape seed oil, jojoba seed oil, sweet almond oil,; polyols; and other ingredients such as hydrolysed wheat and/or soy protien amino acids, rice bran wax, hydroxyethyl behenamidopropyl dimonium chloride, aloe leaf extract, aloe barbadensis leaf juice, phytantriol, retinyl palmitate, behentrimonium methosulphate, cyclopentasiloxane, quaternium-91, stearamidopropyl dimethylamine that improve the appearance of hair, for example by increasing sheen and silky feel, decreasing static electricity, increasing smoothness.

In the present invention, a “conditioning composition” is a composition containing an effective amount of one or more conditioning agents, which is applied to hair to soften, smooth or change the sheen of the hair.

The conditioning composition, or conditioner, can be of any of the known types, namely pack, leave-in, ordinary and hold.

Preferably, according to this preferred embodiment (i.e. in the second method), step (b) is carried out for 5 to 10 minutes, more preferably 8-12 minutes.

Preferably, in step b) of the second method, the keratin-containing tissue, more preferably human or animal hair, even more preferably human scalp hair, is exposed to the light for 5-15 minutes, more preferably 8-12 minutes.

Preferably, according to this preferred embodiment, step c) is preceded by a step c′) of leaving the keratin-containing tissue, more preferably human or animal hair, even more preferably human scalp hair, to process without exposure to radiation for at least 5 minutes, more preferably 10 to 45 minutes, even more preferably 20 to 40 minutes.

Preferably, the first method of the present invention is repeated after a time comprised between 1 week and 6 months, more preferably between 1 and 3 months.

In order to obtain optimal results, the second method of the present invention can be carried out at a frequency of 1-20 times a month, more preferably 1-4 times a week, most preferably 1-2 times a week, more preferably for 4-8 weeks.

After the treatment according to the first method of the present invention, it is in fact good practice to repeat the method (i.e. carry out the second method of the invention) one or two months after the previous treatment. One repetition is usually sufficient to maintain the desired results. However, the second method can be repeated more than once, and at a higher frequency, depending on the results to be achieved.

Preferably, according to this preferred embodiment, in the second method, the keratin-containing tissue is human or animal hair, even more preferably human scalp hair, and it is exposed to light using the radiation-emitting comb or radiation-emitting brush.

In the following, the expressions “radiation-emitting comb” and “radiation-emitting brush” shall be used interchangeably.

In a further preferred embodiment of the present invention, steps a) to c) of the first method of the present invention are repeated and the cosmetic composition has a concentration of reactive molecules that is lower than the concentration of the cosmetic composition in the first method and/or the second method.

In the following, the repetition of the first method of the present invention according to this further preferred embodiment will also be referred to as the “third method”.

The third method of the present invention can be repeated once or twice a week, more preferably for 1-8 weeks.

Preferably, according to this further preferred embodiment, in the third method, the keratin-containing tissue, more preferably human or animal hair, even more preferably human scalp hair, is exposed to light using a radiation-emitting comb or is exposed to visible light.

The third method, in fact, is particularly suited for being performed at home.

Preferably, the third method of the invention follows the first method and/or the second method of the invention.

Preferably, according to this further preferred embodiment (third method), the cosmetic composition comprises at least one conditioning agent.

The present invention also refers to said cosmetic composition comprising said reactive molecules and at least one photoinitiator.

The cosmetic composition is stable for at least six months, preferably at least 12 months, even more preferably, at least 2 years, upon storage at a temperature comprised between 4 and 25° C. By “stable”, what is meant is that more than 30% of the molecules are intact and reactive to light in the presence of a suitable photoinitiator.

The present invention further refers to the use of the cosmetic composition of the present invention on keratin-containing tissue, preferably human or animal hair, human nails and/or human or animal skin, more preferably human or animal hair, even more preferably human scalp hair, comprising at least two free thiol groups for the treatment and/or prevention of damaged keratin.

The present invention further refers to the use of the cosmetic composition of the present invention on keratin-containing tissue, preferably human or animal hair, human nails and/or human or animal skin, more preferably human or animal hair, even more preferably human scalp hair, comprising at least two free thiol groups for the formation of a crosslink between the at least two free thiol groups (that is, the formation of a disulphide bridge). The crosslink is the result of a covalent bond between the at least one light reactive functional group of the reactive molecules and the free thiols.

The present invention further refers to the use of the cosmetic composition of the present invention on human or animal hair, even more preferably human scalp hair, comprising at least two free thiol groups, for closing the hair cuticles.

Preferably, said keratin-containing tissue is damaged keratin-containing tissue.

The present invention further refers to the use of the composition of the present invention on keratin-containing tissue, preferably human or animal hair, more preferably human scalp hair, comprising keratin filaments covalently bound to the reactive molecules.

Preferably, the keratin-containing tissue, preferably human or animal hair, more preferably human scalp hair, comprising keratin filaments covalently bound to the reactive molecules has previously undergone the method for the treatment and/or prevention of damaged hair of the present invention.

Preferably, the cosmetic composition of the present invention is used in association with exposure of the keratin-containing tissue to light.

Preferably, the cosmetic composition of the present invention is used in association with another cosmetic product, such as for example a hair conditioning composition, a hair dyeing/colouring product or a hair spray, as well as hand creams or body creams.

The cosmetic composition of the present invention may also be a hair-bleaching, -straightening, -waving, -curling or -colouring formulation.

The reactive molecules of the cosmetic composition are in fact compatible with other molecules for various hair treatments.

The present invention also refers to the use of a colouring agent, preferably a fluorescent colouring agent, more preferably covalently bound to the reactive molecules, in the cosmetic composition of the present invention for the visualisation of the evenness of the application of the cosmetic composition of the invention. In particular, the colouring agent can be detected upon exposure of the keratin-containing tissue to a light having a suitable wavelength.

In addition or alternatively, the colouring agent as described above can also be used to permanently colour the keratin-containing tissue.

The method of the present invention provides a number of advantages.

The major advantage of the method of the present invention is that it has a long lasting effect.

The optimal result is enhanced by the fact that covalent binding to the keratin occurs solely at the time of exposure to radiation. In the cases where it applies, following rinsing of the keratin-containing tissue, with removal of all residues, including the photoinitiator, the covalently bound molecules do not further react with radiation. The result obtained is thus consistent in time and the method is safe.

The covalent bond between the reactive molecules and the free thiols, as well as repairing the damage on the keratin filament, also provides a protective layer against future mechanical and chemical stress. Such protective layer can make the keratin-containing tissue look healthier, and in the case of hair it can enhance the volume of the hair, making it look even healthier.

In addition, the protective layer can have a moisturising effect, thus preventing for example dry hair and dry skin.

Moreover, the reactive molecules of the invention, as well as any other ingredient in the composition used in the method of the present invention can be chosen to be organic and of natural origin. As a consequence, the composition can be environmentally friendly and safe to handle.

The reactive molecules of the present invention are particularly stable upon storage. This is because the functional groups are covalently bound. This allows long storage times of the composition.

The method of the present invention is also easy to carry out and does not require expensive equipment.

It can be carried out by professionals (for example professional hair-stylists) but can also be adapted to be carried out at home, for example with the use of a UV-comb, a laser comb, or by exposure of the hair to visible light during the processing time.

Accordingly, the present invention refers to a kit of parts for the treatment and/or prevention of damaged keratin in human or animal hair, preferably human scalp hair, comprising a first and a second formulation of the cosmetic composition comprising reactive molecules of the invention.

At least one of the first and second formulation can comprise a conditioning agent. Alternatively, at least one of the first and second formulation can be added to a conditioning composition.

The first formulation is a composition suitable for the repetition of the method of the invention on hair that comprises the reactive molecules (i.e. the second method of the present invention). The second formulation has a lower concentration of reactive molecules than the first formulation, and can be used once or twice a week. The second formulation is suitable for carrying out the third method of the present invention.

The first and second formulation may be a bleaching, straightening, waving, curling or coloring formulation.

The kit may also comprise a third formulation of the cosmetic composition, for use in the method of the invention, wherein the composition has a concentration of reactive molecules higher or the same as the first formulation and is suitable to be applied on damaged hair, preferably that does not comprise the reactive molecules on the keratin filaments thereof. The third formulation is thus suitable for carrying out the first method of the present invention.

In a preferred embodiment, the cosmetic composition comprises at least one photoinitiator that is activated in the visible spectrum.

In an alternative preferred embodiment, the cosmetic composition comprises at least one photoinitiator that is activated in the UV spectrum.

The kit may also comprise a radiation-emitting comb.

### BRIEF DESCRIPTION OF THE FIGURES

FIG. 1 is a schematic representation of keratin filaments of hair. FIG. 1a shows the effect of keratin damage on the disulphide bonds between keratin filaments. FIG. 1b shows the effect of the method of the present invention on damaged keratin where the functional groups on the reactive molecules are acrylates or methacrylates. FIG. 1c shows the effect of the method of the present invention on damaged keratin where the functional groups on the reactive molecules are alkynes.

FIG. 2 shows electron microscopic images of DTT damaged hair (2a) and repaired hair following the treatment method of the invention (2b).

FIG. 3 shows two photographs comparing a volunteer's hair before (3a) and after (3b) treatment with the method of the invention.

FIG. 4 shows a photograph of hair irradiated with UV light following application of the cosmetic composition in the treatment of the invention, wherein the composition contains a fluorescent dye.

### DETAILED DESCRIPTION OF THE INVENTION

The present invention will be further described making reference to the examples provided hereafter by way of illustrative and non-limiting cases.

### Example 1

### Preparation of a Composition Comprising Methacrylate-Functionalised Gelatin.

20% w/v fish gelatin (Weishardt International, France) was dissolved in distilled water to 500 ml at 55° C. The pH was adjusted to 8.5-9 using 3N Sodium Hydroxide (Sigma). Methacrylic anhydride (MAA, obtained from Sigma-Aldrich (Sweden) was added to the solution, with continuous vigorous stirring, in 3 molar excess with respect to gelatin lysine side groups (i.e. at a ratio of 1:3 of the mole percent of gelatin lysines to methacrylic anhydride) without altering the pH and the temperature to obtain the functionalisation of the gelatin with methacrylic groups on the side chains. After 1 hour of reaction, the mixture was dialysed against water (pH >8-9) for 24 hours using Dialysis Membranes (MWCO 12000-14000 kD) obtained from Spectrum Laboratories, Inc. (CA, US) at room temperature (about 25° C.), to remove any unreacted methacrylic anhydride and other side products of the reaction.

The reaction product was then freeze-dried. The degree of substitution (DS) was determined by NMR. Analyses by NMR showed a higher than 50% modification of the gelatin. In other words, more than 50% lysine amines had been replaced with methacrylate groups.

### Example 2

### Preparation of a Composition Comprising Acrylate-Functionalised Gelatin.

The method of Example 1 was carried out by replacing methacrylic anhydride with acrylic anhydride (MP Biomedicals, USA). Analyses by NMR showed more than 55% modification of the gelatin.

### Example 3

### Preparation of a Composition Comprising Glycidyl Methacrylate-Functionalised Gelatin.

The method of Example 1 was carried out by replacing methacrylic anhydride with glycidyl methacrylate (Sigma-Aldrich, Sweden)) where the pH of the solution was adjusted to 3.5-4 using 1M HCl. After this, 6 mL of glycidyl methacrylate was added into the solution dropwise. The reaction was conducted at 50° C. for 24 h and the product was purified by dialysis against Milli-Q water (Millipore Corporation, Darmstadt, Germany) with the above-mentioned dialysis membrane at 40° C. for five days. Analyses by NMR showed more than 40% modification of the gelatin.

### Example 4

### Preparation of a Composition Comprising Vinyl Benzene-Functionalised Gelatin.

The method of Example 1 was carried out by replacing methacrylic anhydride with 4-vinyl benzene chloride (VBC) (Sigma-Aldrich, Sweden) solubilised in 0.5M hydrochloric acid and the solution pH was neutralized to pH 7.4-8 before adding VBC in molar excess at a molar ratio of 1:5 with respect to the lysine amines in gelatin (i.e. at a ratio of 1:5 of the mole percent of gelatin lysines to VBC). The reaction was conducted at 50° C. for 24 h and the product was purified by dialysis against Milli-Q water (Millipore Corporation, Darmstadt, Germany) with the above-mentioned dialysis membrane at 40° C. for five days. Analyses by NMR showed a >30% modification of the gelatin.

### Example 5

### Preparation of a Composition Comprising Alkyne-Functionalised Gelatin.

The method of Example 1 was carried out by replacing methacrylic anhydride with propargyl N-hydroxysuccimide ester (Sigma-Aldrich, Sweden), solubilised in minimal quantity dimethyl sulfoxide (DMSO) (Sigma), in molar excess at a 1:5 molar ratio with respect to the mole percent of lysine amine groups in gelatin (i.e. at a ratio of 1:5 of the mole percent of gelatin lysines to propargyl N-hydroxysuccimide ester), that was added drop-wise at 50-55° C. while stirring for 3 hours. The reaction mixture was dialyzed against distilled water (>pH 8) using 12-14 kDa cutoff dialysis tubing (Spectrum Laboratories, Inc., CA, US) for 2 days to remove reaction by-products. Analyses by NMR showed a >25-30% modification of the gelatin.

### Example 6

### Preparation of a Hair Composition.

A 5% (w/v) composition of the product of Example 1 was prepared by dissolving appropriate amounts in distilled water mixed with 1% of photoinitiator Irgacure 2959® (supplied by Sigma Aldrich, Sweden) that had been previously dissolved in hot water at 80° C. Preservatives including Sodium benzoate and phenoxyethanol were also added as antimicrobial agents. The composition exhibited a shelf life of >12 months.

### Example 7

### Hair Treatment (First Method of the Invention)

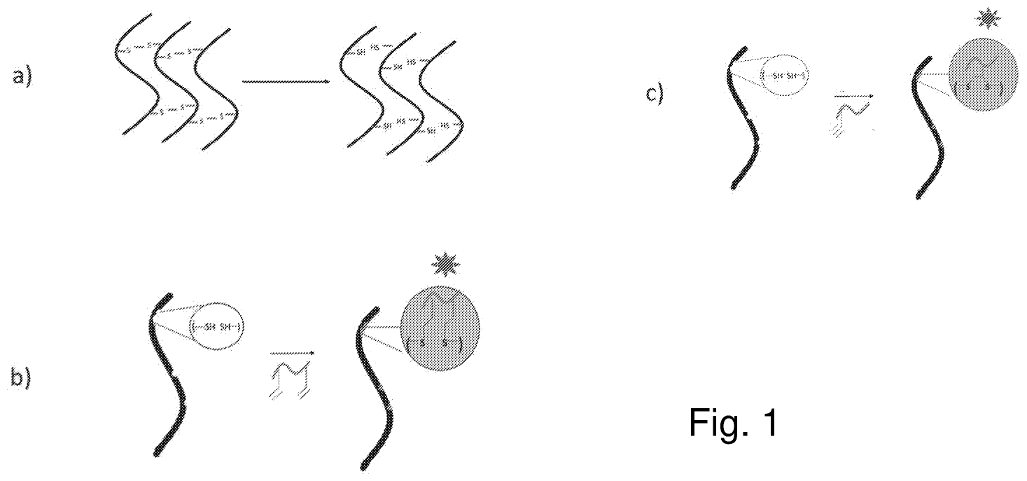

Healthy hair from four volunteers was damaged by applying dithiotretiol (DTT), 200 mM, dissolved in phosphate buffered saline (PBS, pH 7.4) in an amount sufficient to cover all the hair, for two hours. The action of DTT is schematically represented in FIG. 1b The composition of Example 6 was applied on the damaged hair with combing, while shining ultraviolet light (385-400 nm) for 15 minutes using a Dibotech UV flashlight (Kjell & Company, Sweden). The composition was then rinsed off the hair with water and the hair was dried. The action of the composition is schematically represented in FIG. 1b.

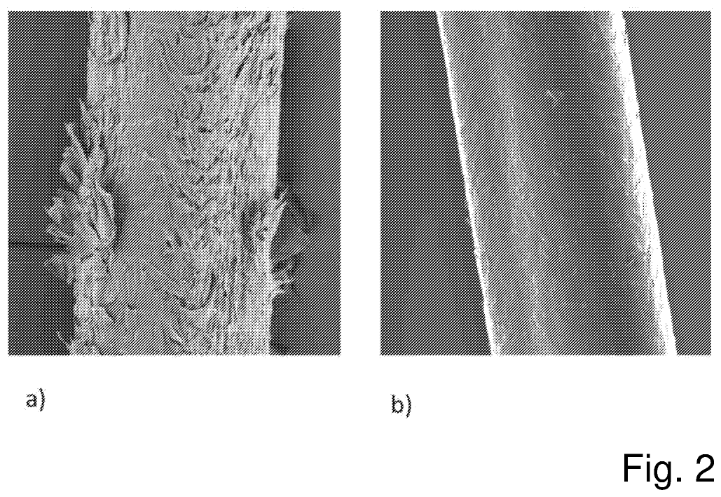

An electron microscopic image was imaged of the hair after treatment using Scanning Electron Microscope. This was compared with a previously obtained image of DTT damaged hair. The results are shown in FIG. 2, where a comparison is shown between a damaged hair following DTT exposure (FIG. 2a) and a repaired hair following the treatment method of the invention (FIG. 2b). It can be seen that whereas in FIG. 2a, the cuticle has a rough and uneven surface, in FIG. 2b, the cuticle is smooth.

The appearance of the hair was also visibly glossier and shinier after the treatment. It also appeared to have increased in volume. The hair following treatment also had a silkier feel to the touch.

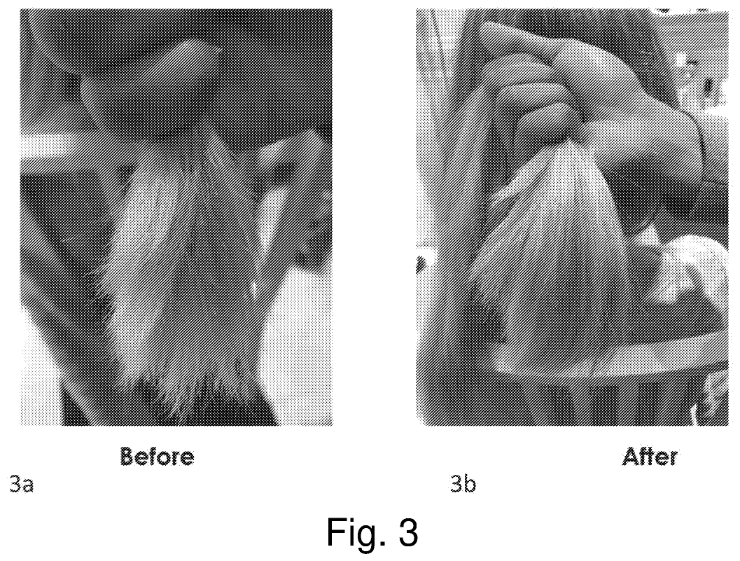

FIG. 3 shows a comparison of the appearance of the hair before (3a) and after (3b) treatment. It can be seen that after the treatment the hair is shinier, more uniform and not as dry.

Upon examination after 4 weeks, it was determined that the hair had maintained a healthy appearance in all volunteers.

### Example 8

### Follow-Up Hair Treatment (Second Method of the Invention)

After the hair treatment of Example 7, a second treatment according to Example 7 was carried out on the same four volunteers with the difference that the composition applied to the hair was a 1:5 (on a weight to weight basis) mixture of the composition of Example and a conditioning composition free of sulfates, phosphates and parabens and having the following composition: Aqua/Water/Eau, Behentrimonium Chloride, Cetearyl Alcohol, Stearamidopropyl Dimethylamine, PPG-3 Benzyl Ether Myristate, PEG-40 Castor Oil, Glycerin, Cetrimonium Chloride, Hydrolyzed Vegetable Protein PG-Propyl Silanetriol, Argania Spinosa (ARGAN) Kernel Oil, Simmondsia Chinensis (Jojoba) Seed Oil, Persea Gratissima (Avocado) Oil, Keratin Amino Acids, Hydrolyzed Keratin, Sodium PCA, Silk Amino Acids, Rosmarinus Officinalis (Rosemary) Leaf Extract, Lavandula Angustifolia (Lavender) Flower Extract, Chamomilla Recutita (Matricaria) Flower Extract, Amodimethicone, Trideceth-12, C11-15 Pareth-7, Laureth-9, Panthenol, Isopropyl Alcohol, Propylene Glycol Dicaprylate/Dicaprate, PPG-1 Trideceth-6, Hydroxypropyl Guar Hydroxypropyltrimonium Chloride, Polyquaternium-37, Propylene Glycol, Cinnamidopropyltrimonium Chloride, Disodium EDTA, Fragrance (Parfum), Phenoxyethanol, Ethylhexylglycerin, Citric Acid, Yellow 5 (CI 19140), Red 33 (CI 17200), Butylphenyl Methylpropional, Hydroxyisohexyl 3-Cyclohexene Carboxaldehyde, Linalool, Alpha-Isomethyl Ionone.

After the application of the composition, the hair was left to process for 10 minutes with exposure to radiation, as in Example 7. After the processing time, the hair was left to rest for a further 10 minutes, without exposure to radiation. The hair was then thoroughly rinsed.

Upon examination it was determined that the hair had a healthy appearance on all volunteers.

### Example 9

### Follow-Up Conditioning (Third Method of the Invention)

The volunteers were given a 100 ml bottle of a 1:5 (w/w) mixture of a composition prepared according to Example 6, with the difference that the reactive molecules were present in a 0.8% (w/v) concentration with EosinY; and the conditioning composition described in Example 8.

The volunteers were instructed to use the mixture once every two weeks to follow up the treatment from Example 7 and example 8.

The volunteers were instructed to apply the conditioner and expose the hair sunlight for 15 minutes, and then rinse off.

At the end of the 8 weeks, the hair of the volunteers was examined. The hair had maintained a healthy appearance in all volunteers.

### Example 10

### Hair Composition Containing a Fluorescent Dye

A composition was prepared according to Example 6 with the difference that Ebest Fluorescein LTC (purchased from Fastcolors, UK) was also added to the mixture (in a concentration in weight percent based on the volume of the total composition of 0.001%).

The composition was then applied to the hair of a volunteer, according to Example 7.

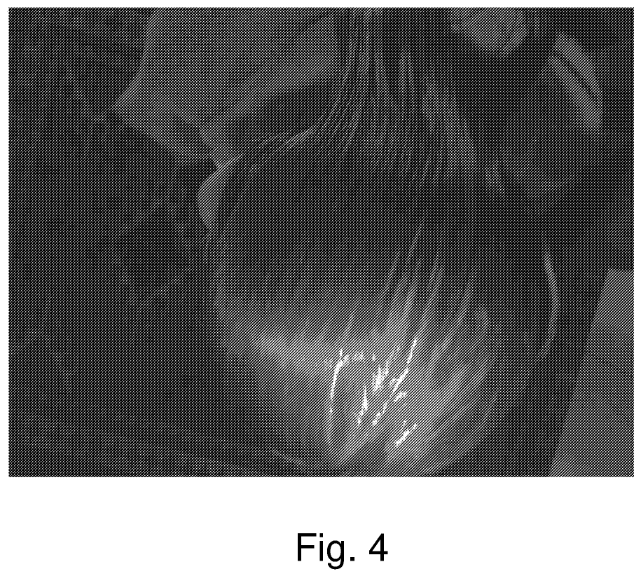

It was observed that, as the UV light (385-400 nm) was shined on the hair, the hair composition illuminated, making it possible to detect any areas of the hair where the composition had not been applied. See FIG. 4.

### Example 11

### Preparation of a Composition Comprising Alkene-Functionalised Hydrolysed Soy Protein.

5%(w/v) hydrolysed soy protein powder (Kelisema, Italy) was dissolved in Milli Q water to 100 ml and gently stirred. The pH was adjusted to 7.5-8.5 using 2N NaOH (Sigma). Subsequently, methacrylic anhydride (Sigma-Aldrich, Sweden) was added to the solution, drop-wise at room temperature (25° C.) at a molar ratio of 5:1 with respect to the mole percent of lysine amine groups in the soy protein to modify the hydrolysed soy protein with reactive methacrylate functional groups.

After 1 hour of reaction, the mixture was dialysed against water (pH >8-9) for 24 hours using Dialysis Membranes (MWCO 100-500D) obtained from Spectrum Laboratories, Inc. (CA, US) at room temperature (about 25° C.), to remove any unreacted methacrylic anhydride and other side products of the reaction.

The reaction product was then freeze-dried. The degree of substitution (DS) was determined by NMR by comparison of the pristine hydrolysed soy protein and hydrolysed soy protein methacrylate.

Analyses by NMR showed a higher than 50% modification of the hydrolysed soy protein with methacrylic groups.

### Example 12

### Preparation of a Composition Comprising Alkyne-Functionalised Hydrolysed Soy Protein.

5%(w/v) hydrolysed soy protein powder (Kelisema, Italy) was dissolved in Milli Q water to 100 ml and gently stirred. The pH was adjusted to 7.5-8.5 using 2N NaOH (Sigma). Subsequently, propargyl N-hydroxysuccimide ester (Sigma-Aldrich, Sweden) solubilised in minimal quantity dimethyl sulfoxide (DMSO, Sigma) was added to the solution, drop-wise, while stirring for three hours at room temperature (25° C.) in molar excess at a 1:5 molar ratio with respect to the mole percent of lysine amine groups in hydrolysed soy protein (i.e. at a ratio of 1:5 of the mole percent of hydrolysed soy protein lysines to propargyl N-hydroxysuccimide ester) to modify the hydrolysed soy protein with reactive methacrylate functional groups.

After 1 hour of reaction, the mixture was dialysed against water (pH >8-9) for 24 hours using Dialysis Membranes (MWCO 100-500D) obtained from Spectrum Laboratories, Inc. (CA, US) at room temperature (about 25° C.), to remove any unreacted methacrylic anhydride and other side products of the reaction.

The reaction product was then freeze-dried. The degree of substitution (DS) was determined by NMR by comparison of the pristine hydrolysed soy protein and hydrolysed soy protein methacrylate.

Analyses by NMR showed a higher than 50% modification of the hydrolysed soy protein with methacrylic groups.

1-26. (canceled)

27. A method for treating and/or preventing damaged keratin in a keratin-comprising tissue, the method comprising:
a) applying on the keratin-comprising tissue a cosmetic composition comprising a first molecule comprising a light-reactive functional group, and a photoinitiator; and
b) allowing exposure of the keratin-comprising tissue to light, the light being of a wavelength suitable for activating the photoinitiator.

28. The method of claim 27, wherein the keratin-comprising tissue comprises human hair, animal hair, human skin, animal skin, and/or human nails.

29. The method of claim 27, wherein the keratin-comprising tissue comprises human hair and/or animal hair.

30. The method of claim 27, wherein the keratin-comprising tissue comprises human scalp hair.

31. The method of claim 27, wherein the light-reactive functional group comprises an acrylate, methacrylate, vinyl benzene, alkyne, or combination of two or more of any of these.

32. The method of claim 27, wherein the first molecule is obtained by a process comprising functionalizing an aminoacid, saccharide, fatty acid, residue thereof, or a combination of two or more of any of these.

33. The method of claim 27, wherein the first molecule is obtained by a process comprising functionalizing a synthetic polymer.

34. The method of claim 27, wherein, in the cosmetic composition in the applying a), the first molecule is present in a concentration, in weight percent based on a volume of the cosmetic composition, of at least 0.01% (w/v).

35. The method of claim 27, wherein, in the cosmetic composition in the applying a), the first molecule is present in a concentration in a range of from 0.05 to 20% (w/v).

36. The method of claim 27, wherein, in the cosmetic composition in the applying a), the first molecule is present in a concentration in a range of from 0.1 to 15% (w/v).

37. The method of claim 27, wherein, in the cosmetic composition in the applying a), the first molecule is present in a concentration in a range of from 0.5 to 10% (w/v).

38. The method of claim 27, wherein the photoinitiator is activated at a wavelength in a range of from 10 nm to 100 μm.

39. The method of claim 27, wherein the photoinitiator is activated at a wavelength in a range of from 300 to 1400 nm.

40. The method of claim 27, wherein the photoinitiator is activated at a wavelength in a range of from 360 to 800 nm.

41. The method of claim 27, wherein the photoinitiator is activated at a wavelength in a range of from 360 to 400 nm.

42. The method of claim 27, wherein the cosmetic composition further comprises:
a coloring agent.

43. The method of claim 42, wherein the coloring agent is capable of emitting light when exposed to radiation having a wavelength in a range of from 10 nm and 100 μm.

44. The method of claim 27, wherein the cosmetic composition further comprises:
a pigment, a dye, or a combination of two or more of any of these.

45. A cosmetic composition, comprising:
a molecule comprising a light-reactive functional group; and
a photoinitiator.

46. A kit of parts suitable for treating and/or preventing damaged keratin in human or animal hair, the kit comprising:
a first formulation comprising a first form of the cosmetic composition of claim 45; and
a second formulation comprising a second form of the cosmetic composition, different from the first form.
